(19) United States
(12) Patent Application Publication
(10) Pub. No.: US 2022/0331136 A1
(43) Pub. Date: Oct. 20, 2022

(54) Adjustable Posture Resistance Band

(71) Applicant: Megan Ivanoff, Grafton, MA (US)

(72) Inventor: Megan Ivanoff, Grafton, MA (US)

(21) Appl. No.: 17/723,426

(22) Filed: Apr. 18, 2022

Related U.S. Application Data
(60) Provisional application No. 63/176,011, filed on Apr. 16, 2021.

(51) Int. Cl. A61F 5/02

(52) U.S. Cl. CPC A61F 5/026

(57) ABSTRACT

An adjustable posture resistance band is an apparatus that is used to strengthen posture with wear and exercise. The apparatus includes a left trap band, a right trap band, at least one ring, a left abdominal strap, and a right abdominal strap. The left trap band and the right trap band wrap around the shoulders creating tension against which the user can work to strengthen postural musculature. The at least one ring connects the left trap band with the right trap band. The left abdominal strap and the right abdominal strap wrap around the back to the abdominal region to safely position the trap bands in the axilla and enhance comfort. The left trap band and the right trap band are each length adjustable from the shoulders to the chest with a fastener mechanism. The left abdominal strap and the right abdominal strap are each length-adjustable around the abdominal region.

The current application claims a priority to the U.S. Provisional Patent application Ser. No. 63/176,011 filed on Apr. 16, 2021. The current application is filed on Apr. 18, 2022 while Apr. 16, 2022 was on a weekend.

### FIELD OF THE INVENTION

The present invention generally relates to exercise equipment. More specifically, the present invention is an adjustable posture resistance band.

### BACKGROUND OF THE INVENTION

Maintaining good posture is something that many people struggle with. For the average person, a majority of daily activities including computer and mobile use are performed in a forward flexed and forward shoulder position, leading to poor posture and a plethora of pain syndromes and injuries. Unfortunately, the advice most people get and devices available to counter this bad habit are inadequate to achieve long term postural re-education.

Good posture is maintained by the muscular system which must be strengthened and trained to support spinal alignment throughout the day. Many people utilize a postural brace to improve their posture, but by doing so are assisting their postural musculature. This approach is counterproductive. A muscle does not become stronger when it is consistently assisted. To improve strength and endurance, muscles must work safely against resistance.

There are currently no resistance devices that safely and adequately provide resistance at the posterior shoulder to aid in improving endurance of postural musculature through full upper extremity range of motion. Most people perform postural exercises by holding weights or resistance band or cables, however two issues arise when training in this way. First, as the distance and number of joints increases between a resistance point and the targeted muscle, there is a higher risk of compensatory patterns, poor form and injury. Second, performing short bursts of exercise cannot adequately train endurance of postural muscles to maintain good posture for hours throughout the day. There is a need for a resistance device that can safely anchor at the shoulder without risk of neurovascular compression in the axilla, to strengthen shoulder retraction and thoracic extension for extended periods of time throughout an entire workout routine or during daily activities. Such a device should remain in place throughout full shoulder range of motion so as to not limit exercises to which it may be applied. By applying a low load long duration (LLLD) resistance to the shoulders, such a device can help increase postural endurance, resulting in improved spinal alignment throughout the day to minimize pain and injuries related to poor posture.

### BRIEF DESCRIPTION OF THE DRAWINGS

FIG. 1 is a front side view of the present invention in a preferred configuration around a user.

FIG. 2 is a rear side view of the present invention in the preferred configuration around a user.

FIG. 3 is a front perspective view of the present invention with a second brace end of a left trap brace and a second brace end of a right trap brace disengaged with at least one ring.

FIG. 4 is a magnified front side view of a first brace end of the left trap band of the present invention.

FIG. 5 is a front side view of the present invention with a left ring and a right ring in the preferred configuration around a user.

FIG. 6 is a front side view of the present invention with both the left trap brace and the right trap brace engaged with the at least one ring.

FIG. 7 is a rear perspective view of the present invention with the second brace end of the left trap brace and the second brace end of the right trap brace disengaged with at least one ring.

FIG. 8 is a front side view of the present invention in an alternate configuration around a user with a left abdominal strap attached with a first linear portion of the right trap brace, a right abdominal strap attached with a first linear portion of the left trap brace, and a resistance band connected with the at least one ring.

### DETAILED DESCRIPTIONS OF THE INVENTION

All illustrations of the drawings are for the purpose of describing selected versions of the present invention and are not intended to limit the scope of the present invention.

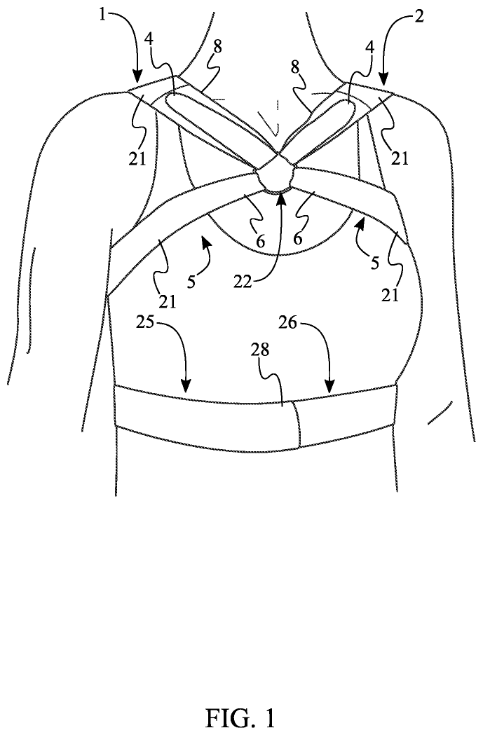

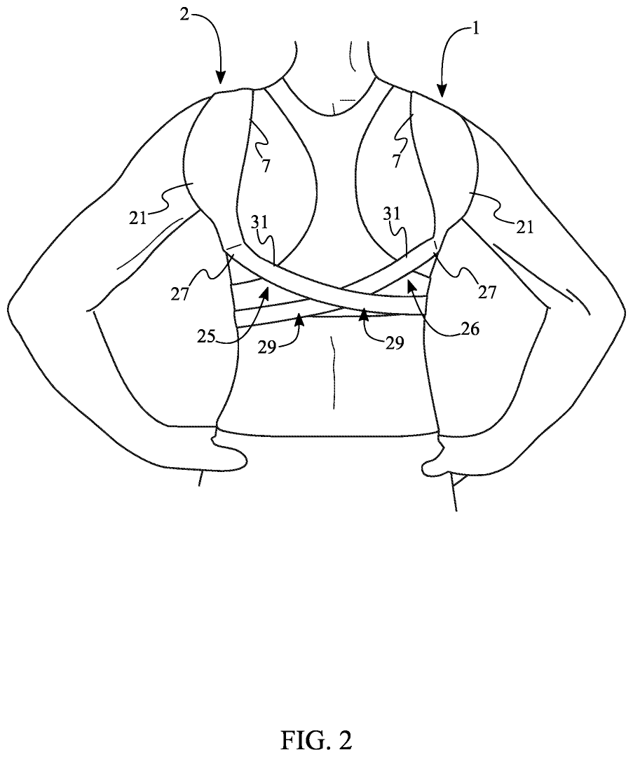

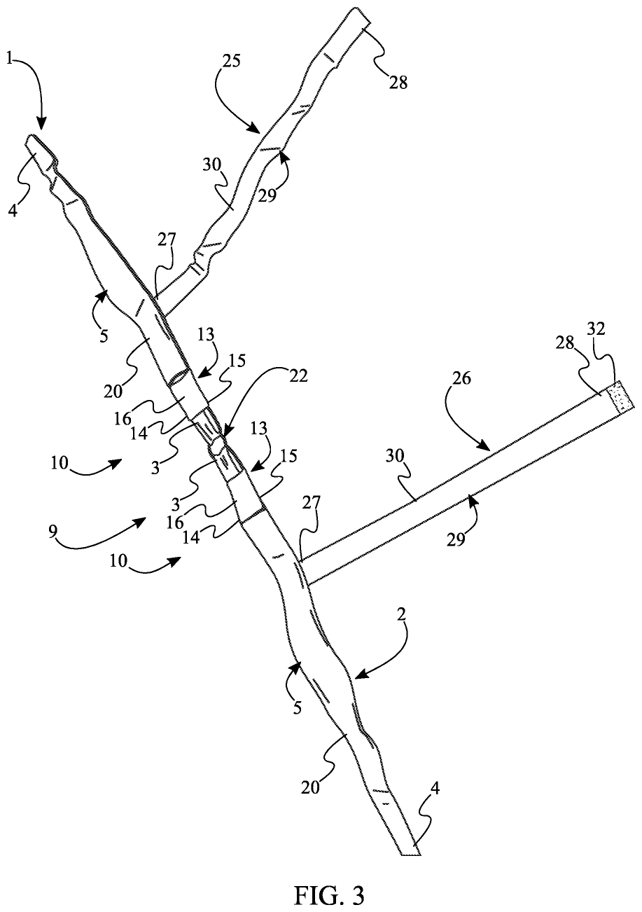

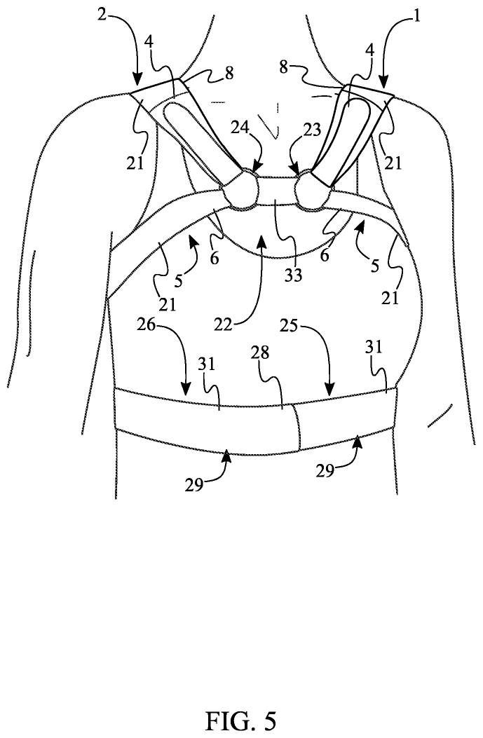

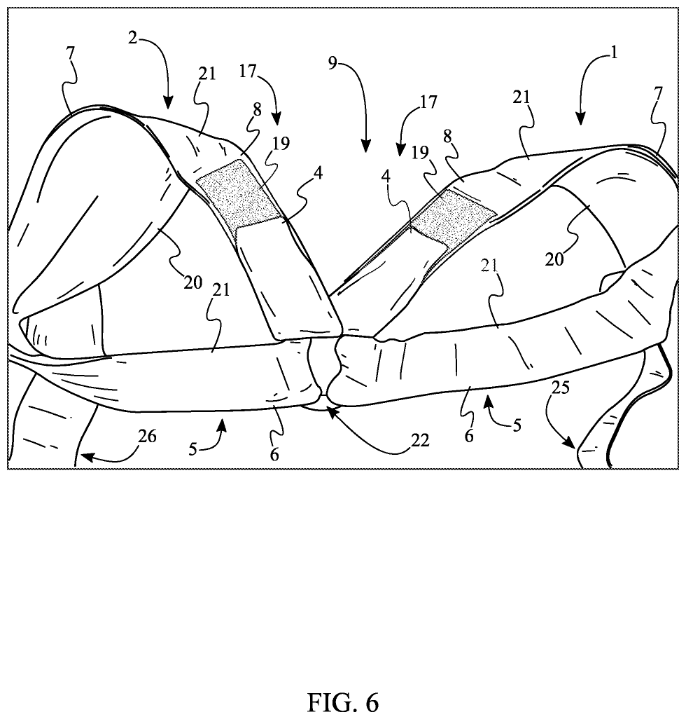

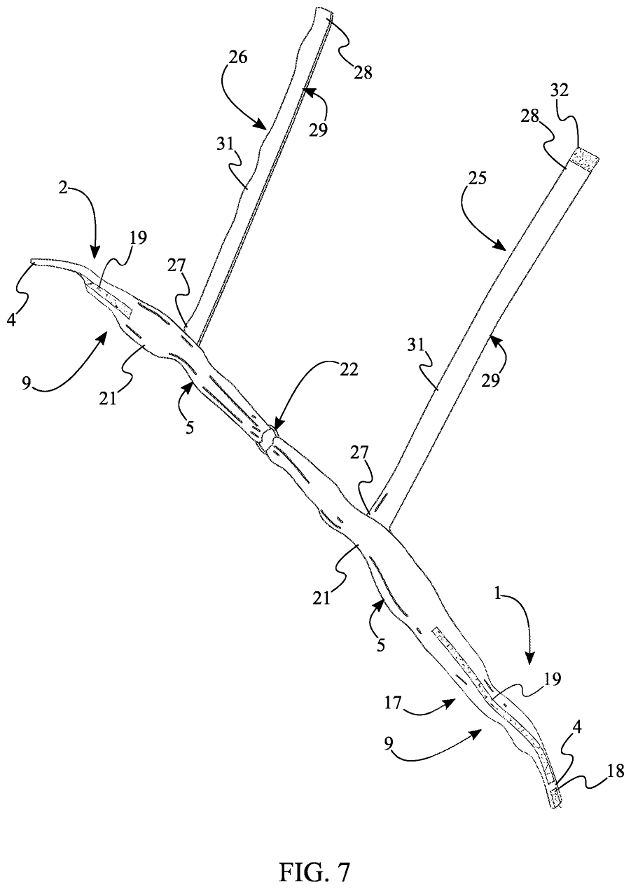

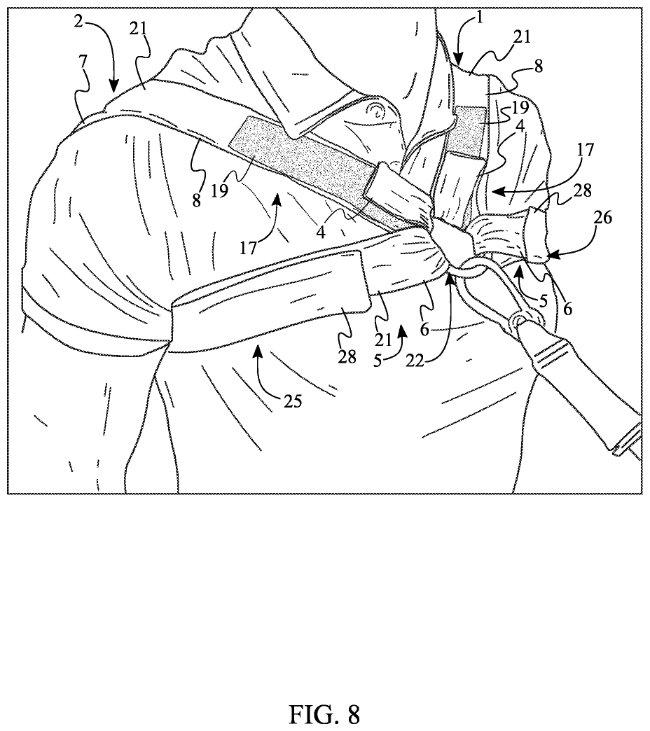

The present invention is an adjustable posture resistance band. The present invention trains the upper body muscles of a user for correct posture. More specifically, the present invention trains the upper body muscles throughout wear and exercise. The present invention wraps around the back of the shoulders, across the traps, under the armpits and across the chest. Additionally, the present invention also wraps around the abdominal region of the user for stability. The present invention is adjustable to the shape and size of the user. The present invention may be engaged with a resistance band or cable to increase the intensity of the exercise being performed. Moreover, the present invention provides tactile feedback which encourages the shoulders of a user to be lowered and pulled back for both correct posture and comfortable stance. The present invention encourages the user to actively retract the shoulders while wearing the present invention. In order to effectively engage the upper body muscles that define correct posture, the present invention comprises a left trap band 1, a right trap band 2, at least one ring 22, a left abdominal strap 25, and a right abdominal strap 26, seen in FIG. 1, FIG. 2, FIG. 3, FIG. 5, FIG. 6, FIG. 7, and FIG. 8. The left trap band 1 and the right trap band 2 pull the shoulders forward when tightened or engaged with a resistance band or cable so that the user must push back against the left trap band 1 and the right trap band 2 to both engage and strengthen the muscles needed for correct posture. It is understood that a free end of the resistance band or cable is connected with a fastener such as a carabiner or clip and a fixed end is mounted onto a structure or surface. Furthermore, exercising with the present invention while connected with a resistance band or cable, the user strengthens musculature that retracts the shoulders of the user and extends the spine to improve postural endurance and maintain optimal spinal alignment throughout the day. Additional exercises that may be performed while wearing the present invention to encourage correct posture include, but are not limited to, jumping jacks, overhead press, rows, flys, jogging in place, jumping lunges, and bicep and tricep curls. In order to wrap around each shoulder of the user, the left trap band 1 and the right trap band 2 each comprise a first band end 3, a second band end 4, a band body 5, and a fastener mechanism 9. The first band end 3 and the second band end 4 connect the band body 5 with the at least one ring 22. The band body 5 can be tightened to increase resistance while only wearing the present invention, as well as for resistance while exercising with the present invention. The fastener mechanism 9 allows the first band end 3 and the second band end 4 to be fastened around the at least one ring 22. The at least one ring 22 serves as a point of connection between the left trap band 1 and the right trap band 2. Furthermore, the at least one ring 22 serves as a point of force away from the chest of the user while a user exercises with the present invention, specifically while engaged with a resistance band or cable. In order for the present invention to be engaged around the abdominal region of the user, the left abdominal strap 25 and the right abdominal strap 26 each comprise a first strap end 27, a second strap end 28, and a strap body 29. The first strap end 27 connects the strap body 29 with the left trap band 1 and the right trap band 2, respectively. The strap body 29 presses against the abdominal region of the user. The second strap end 28 secures the strap body 29 around the abdominal region.

The overall arrangement of the aforementioned components allows the present invention to be effectively secured around the bodies of a variety of users. The present invention is centered with the body of the user as the at least one ring 22 is positioned in between the left trap band 1 and the right trap band 2. Moreover, as seen in FIG. 1 and FIG. 8. the present invention is easily centered with the body of the user by aligning the at least one ring 22 in the middle of the chest. The first band end 3 and the second band end 4 are positioned opposite to each other along the band body 5, thereby looping around the corresponding shoulder. In order for the left trap band 1 to be tightened accordingly around the left shoulder of the user, the first band end 3 of the left trap band 1 and the second band end 4 of the left trap band 1 are detachably attached to the at least one ring 22 by the fastener mechanism 9 of the left trap band 1. Likewise, the first band end 3 of the right trap band 2 and the second band end 4 of the right trap band 2 are detachably attached to the at least one ring 22 by the fastener mechanism 9 of the right trap band 2. A length of the strap body 29 is defined as the first strap end 27 and the second strap end 28 are positioned opposite to each other along the strap body 29. In order to stabilize present invention around the user and counterbalance the force of the left trap band 1 and the right trap band 2 against the upper body of the user, the first strap end 27 of the left abdominal strap 25 is laterally fixed with the left trap band 1, offset from the at least one ring 22, and the first strap end 27 of the right abdominal strap 26 being laterally fixed with the right trap band 2, offset from the at least one ring 22.

In order to define the preferred circumference of the left trap band 1 around the left shoulder and the right trap band 2 around the right shoulder, the left trap band 1 and the right trap band 2 may each further comprise an inner band surface 20 and an outer band surface 21, seen in FIG. 3 and FIG. 7, and the fastener mechanism 9 may comprise a lower fastener 10 and an upper fastener 17. The inner band surface 20 presses directly against the body of the user. The outer band surface 21 is oriented away from the body of the user and is readily visible to the surrounding environment while being worn by the user. The lower fastener 10 locks and unlocks the first band end 3 with the at least one ring 22. The upper fastener 17 locks and unlocks the second band end 4 with the at least one ring 22. A thickness of the band body 5 is defined as the inner band surface 20 is laterally positioned along the band body 5, and the outer band surface 21 is laterally positioned along the band body 5, opposite to the inner band surface 20. The thickness provides the structural integrity for the left trap band 1 and the right trap band 2 while being worn and especially while utilized during an exercise. The first band end 3 serves as an anchoring point for the left trap band 1 and the right trap band 2, respectively, as the lower fastener 10 is fixed onto the inner band surface 20, adjacent to the first band end 3. This arrangement directs the first end to folded over the at least one ring 22 and under the band body 5. The second band end 4 allows the left trap band 1 and the right trap band 2 to be readily adjustable by the user while being worn as the upper fastener 17 is fixed onto the outer band surface 21, adjacent to the second band end 4. This arrangement directs the second end to be folded under the at least one ring 22 and on top of the band body 5, thereby allowing the second band end 4 to be readily accessed and the left trap band 1 and the right trap band 2, respectively, to be adjusted as needed.

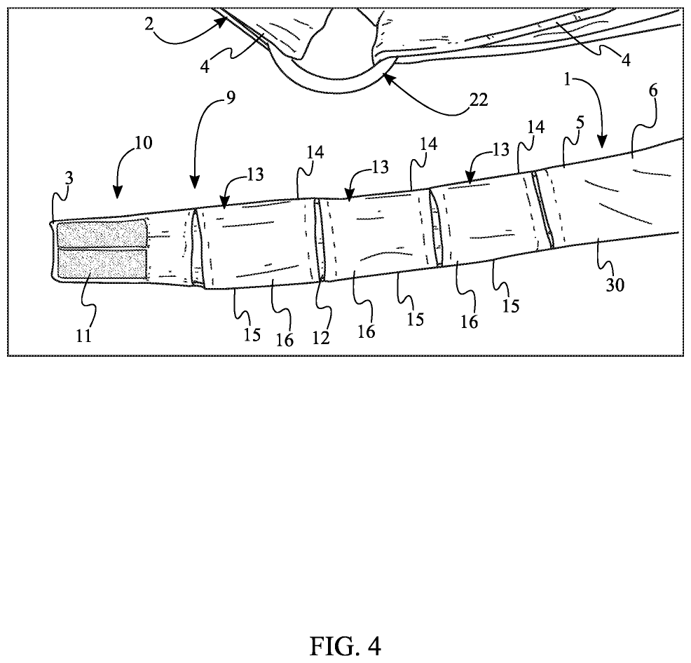

In order to lock and unlock the first band end 3 around the at least one ring 22, the lower fastener 10 may comprise a lower interlocking tab member 11 and a lower interlocking elongated member 12, seen in FIG. 3 and FIG. 4. The lower interlocking tab member 11 connects the first band end 3 at a point along the lower interlocking elongated member 12. The lower interlocking elongated member 12 allows the left trap band 1 and the right trap band 2, respectively, to be length adjustable. The lower interlocking tab member 11 is preferably a hooks portion of a hook-and-loop fastener, and the lower interlocking elongated member 12 is made of an elastic material. Alternatively, the lower interlocking elongated member 12 is a loops portion of a hook-and-loop fastener. The first band end 3 is securely and smoothly attached along the band body 5 as the lower interlocking tab member 11 is fixed adjacent with the first band end 3. This arrangement provides a smooth continuous connection for the first band end 3 around the at least one ring 22 and prevents the first band end 3 from flapping around while looped around the at least one ring 22. Once the first band end 3 has looped over the at least one ring 22 and pulled back towards the band body 5, the lower interlocking elongated member 12 allows the lower interlocking tab member 11 to be positioned along the band body 5 as needed as the lower interlocking elongated member 12 is positioned offset from the lower interlocking tab member 11 and is fixed across the inner band surface 20. More specifically, the lower interlocking tab member 11 of the left trap band 1 is positioned through the at least one ring 22, and the lower interlocking member of the right trap band 2 is positioned through the at least one ring 22 for both the left trap band 1 and the right trap band 2 to be anchored with the at least one ring 22. The left trap band 1 and the right trap band 2 may be adjusted with the first band end 3 as the lower interlocking tab member 11 is detachably attached with the lower interlocking elongated member 12.

In the preferred embodiment of the present invention, the lower fastener 10 further comprises at least one tucking pocket 13, seen in FIG. 4. The at least one tucking pocket 13 prevents the lower interlocking elongated pocket from directly pressing against the skin of the user or the clothes of the user, limiting any aggravation and enhancing comfort. The at least one tucking pocket 13 further ensures a smooth connection between the first band end 3 with the band body 5. In order for the first band end 3 to be positioned in between the at least one tucking pocket 13 and the lower interlocking elongated member 12, the at least one tucking pocket 13 may comprise a first lateral edge 14, a second lateral edge 15, and a pocket panel 16. As the inner band surface 20 presses against the skin or the clothes of the user, the pocket panel 16 is positioned adjacent with the inner band surface 20. More specifically, the pocket panel 16 is positioned across the lower interlocking elongated member 12, thereby shielding the skin or clothes of the user from any abrasion of the first band end 3 along the lower interlocking elongated member 12. The connection between the lower interlocking tab member 11, and consequently the first band end 3, along the lower interlocking elongated member 12 is reinforced with the pocket panel 16 as the first lateral edge 14 and the second lateral edge 15 are positioned opposite to each other across the pocket panel 16. More specifically, the first lateral edge 14 is positioned adjacent and along the lower interlocking elongated member 12. Similarly, the second lateral edge 15 is positioned adjacent and along the lower interlocking elongated member 12, opposite to the first lateral edge 14. The pocket panel 16 remains connected with the band body 5 as the first lateral edge 14 and the second lateral edge 15 are fixed onto the inner band surface 20.

Similar with the first band end 3, in order to secure the second band end 4 around the at least one ring 22, the upper fastener 17 may comprise an upper interlocking tab member 18 and an upper interlocking elongated member 19, seen in FIG. 6, FIG. 7, and FIG. 8. The upper interlocking tab member 18 connects the second band end 4 at a point along the upper interlocking elongated member 19. The upper interlocking elongated member 19 allows the left trap band 1 and the right trap band 2, respectively, to be length adjustable. The upper interlocking tab member 18 is preferably a hooks portion of a hook-and-loop fastener, and the upper interlocking elongated member 19 is made of an elastic material. Alternatively, the upper interlocking elongated member 19 is a loops portion of a hook-and-loop fastener. The second band end 4 is securely and smoothly attached along the band body 5 as the upper interlocking tab member 18 is fixed adjacent with the second band end 4. This arrangement provides a smooth continuous connection for the second band end 4 around the at least one ring 22 and prevents the second band end 4 from flapping around while looped around the at least one ring 22. Furthermore, the connection between the second band end 4 with the band body 5 remains aesthetically pleasing and does not inhibit any workout of the user. Once the second band end 4 has looped under the at least one ring 22 and pulled back towards the band body 5, the upper interlocking elongated member 19 allows the upper interlocking tab member 18 to be positioned along the band body 5 as need as the upper interlocking elongated member 19 is positioned offset from the upper interlocking tab member 18 and is fixed across the inner band surface 20. More specifically, the upper interlocking tab member 18 of the left trap band 1 is positioned through the at least one ring 22, and the upper interlocking tab member 18 of the right trap band 2 is positioned through the at least one ring 22. The left trap band 1 and the right trap band 2 is readily adjustable while being worn by the user as the upper interlocking tab member 18 is detachably attached with the upper interlocking elongated member 19.

In order for the present invention to comfortably grip around the posterior shoulders of the user and comfortably press across the chest of the user, the band body 5 may comprise a first linear portion 6, an ergonomic wider portion 7, and a second linear portion 8, seen in FIG. 1, FIG. 2, FIG. 3, FIG. 5, FIG. 6, FIG. 7, and FIG. 8. The first linear portion 6 and the second linear portion 8 are positioned across the chest, whereas the ergonomic wider portion 7 presses across the posterior shoulders of the user. Moreover, the first linear portion 6 and the second linear portion 8 occupy minimal space across the chest to so that the movements of the user remain uninhibited. Furthermore, the ergonomic wider portion 7 evenly distributes the force against the posterior shoulders of the user for a more comfortable fit for the user while simply wearing the present invention or exercising with it. In a preferred configuration for the present invention the first linear portion 6 extends from the at least one ring 22 to the armpits of the user, whereas the second linear portion 8 extends from the at least one ring 22 towards the shoulders of the user. As the left trap band 1 and the right trap band 2 loop around the shoulders of the user, in order for the ergonomic wider portion 7 to be positioned along the back of the user specifically across the posterior shoulders of the user, the ergonomic wider portion 7 is fixed in between the first linear portion 6 and the second linear portion 8. More specifically, the first band end 3 is positioned coincident with the first linear portion 6, offset from the ergonomic wider portion 7. Likewise, the second band end 4 is positioned coincident with the second linear portion 8, offset from the ergonomic wider portion 7. In order for the left abdominal strap 25 to cross over the right abdominal strap 26 along the backside of the abdominal region, the first strap end 27 of the left abdominal strap 25 is fixed in between the first linear portion 6 of the left trap band 1 and the ergonomic wider portion 7 of the left trap band 1, and the first strap end 27 of the right abdominal strap 26 is fixed in between the first linear portion 6 of the right trap band 2 and the ergonomic wider portion 7 of the right trap band 2. This requires the user cross over the left abdominal strap 25 with the right abdominal strap 26 behind the user, counterbalancing the central point of tension between the left trap band 1 and the right trap band 2 at the at least one ring 22. Furthermore, this arrangement provides support across back of the user and further encourages correct posture. This arrangement also positions the present left trap band 1 and the right trap band 2 around the axilla region of the user and avoids compression of the neurovascular structures.

In order for the left abdominal strap 25 and the right abdominal strap 26 to lock and unlock with each other around the body of the user, the left abdominal strap 25 and the right abdominal strap 26 may each further comprise an inner strap surface 30, an outer strap surface 31, and a strap fastener 32, seen in FIG. 1, FIG. 2, FIG. 3, FIG. 5, FIG. 7, and FIG. 8. The inner strap surface 30 presses against the skin or the clothing of the user. A thickness of the strap body 29 is defined as the inner strap surface 30 is laterally positioned along the strap body 29, and the outer strap surface 31 is laterally positioned along the strap body 29, opposite to the inner strap surface 30. As the second strap end 28 is free, the strap fastener 32 of the left abdominal strap 25 is fixed onto the inner strap surface 30 of the left abdominal strap 25, adjacent to the second strap end 28. Conversely, the strap fastener 32 of the right abdominal strap 26 is fixed onto the outer strap surface 31 of the right abdominal strap 26, adjacent to the second strap end 2. In the preferred embodiment of the present invention, the second strap end 28 of the left abdominal strap 25 and the second strap end 28 of the right abdominal strap 26 being detachably attached with each other, thereby securing the present invention around the abdominal region of the user, regardless of size or shape. In order for the left abdominal strap 25 to be looped around the frontside of the body of the user and tightened to reinforce the backside of the abdominal region of the user, the strap fastener 32 of the left abdominal strap 25 is detachably attached with the outer strap surface 31 of the right abdominal strap 26. The right abdominal strap 26 remains smoothly and securely positioned underneath the left abdominal strap 25 as the strap fastener 32 of the right abdominal strap 26 is detachably attached with the outer strap surface 31 of the left abdominal strap 25. The left abdominal strap 25 and the right abdominal strap 26 may traverse directly underneath the chest of the user, along the waist of the user, or along the lower portion of the stomach of the user along the frontside of the abdominal region. While positioned across the waist of the user, the left abdominal strap 25 and the right abdominal strap 26, together, serve as a tactile cue for the user to engage core musculature, to reduce rib flare, and to reduce over extension of the spine. In a first alternate embodiment of the present invention the right abdominal strap 26 may be overlapped on top of the left abdominal strap 25 and the strap fastener 32 of the left abdominal strap 25, and the strap fastener 32 of the right abdominal strap 26 are fixed with the strap body 29 accordingly such that the right abdominal strap 26 is superimposed across the left abdominal strap 25 as in the preferred embodiment of the present invention.

In a second alternate embodiment of the present invention, seen in FIG. 8, the left abdominal strap 25 and the right abdominal strap 26 are wrapped around the frontside of the user while the abdominal region remains free of the left abdominal strap 25 and the right abdominal strap 26, increasing the range of motion for the user while the backside of the abdominal region remains supported. In this second alternate embodiment, the strap fastener 32 of the left abdominal strap 25 is fixed onto the inner strap surface 30 of the left abdominal strap 25, adjacent to the second strap end 28. Likewise, the strap fastener 32 of the right abdominal strap 26 is fixed onto the inner strap surface 30 of the right abdominal strap 26, adjacent to the second strap end 28. This arrangement orients the strap fastener 32 of the left abdominal strap 25 and the strap fastener 32 of the right abdominal strap 26 towards the right trap band 1 and the left trap band 2. The strap fastener 32 of the left abdominal strap 25 is detachably attached with the outer band surface 21 of the right trap band 2. Similarly, the strap fastener 32 of the right abdominal strap being detachably attached with the outer band surface 21 of the left trap band 1. This arrangement consequently allows the present invention to wrap entirely around the body of the user without the left abdominal strap 25 and the right abdominal strap 26 being directly positioned around the abdominal region of the user.

In the preferred embodiment of the present invention, the at least one ring 22 is a single ring, seen in FIG. 1, FIG. 2, FIG. 3, FIG. 4, FIG. 6, FIG. 7, and FIG. 8. The single ring is preferably a round ring. However, it is understood, that the single ring of alternate embodiments of the present invention may be various shapes. In some other embodiments of the present invention, the present invention further comprises a connecting strap 33, and the at least one ring 22 comprises a left ring 23 and a right ring 24, as seen in FIG. 5. In these other embodiments, the left trap band 1 and the right trap band 2 are further offset from each other for a user with perhaps wider chest dimensions. In order for the left trap band 1 and the right trap band 2 to still remain connected to the at least one ring 22 while not directly attached to the same ring, the left ring 23 is positioned adjacent with the connecting strap 33, and the right ring 24 is positioned adjacent with the connecting strap 33, opposite the left ring 23. The movements of the user remain uninhibited as the left ring 23 and the right ring 24 are tethered to each other by the connecting strap 33. More specifically, similar to the preferred embodiment of the present invention, the first strap end 27 of the left trap band 1 and the second strap end 28 of the left trap band 1 are detachably attached to the left ring 23 by the fastener mechanism 9 of the left trap band 1. Likewise, the first strap end 27 of the right trap band 2 and the second strap end 28 of the right trap band 2 are detachably attached to the right ring 24 by the fastener mechanism 9 of the right trap band 2.

Although the invention has been explained in relation to its preferred embodiment, it is to be understood that many other possible modifications and variations can be made without departing from the spirit and scope of the invention as hereinafter claimed.

What is claimed is:

1. An adjustable posture resistance band comprising:
a left trap band;
a right trap band;
at least one ring;
a left abdominal strap;
a right abdominal strap;
the left trap band and the right trap band each comprising a first band end, a second band end, a band body, and a fastener mechanism;
the left abdominal strap and the right abdominal strap each comprising a first strap end, a second strap end, and a strap body;
the at least one ring being positioned in between the left trap band and the right trap band;
the first band end and the second band end being positioned opposite to each other along the band body;
the first band end of the left trap band and the second band end of the left trap band being detachably attached to the at least one ring by the fastener mechanism of the left trap band;
the first band end of the right trap band and the second band end of the right trap band being detachably attached to the at least one ring by the fastener mechanism of the right trap band;
the first strap end and the second strap end being positioned opposite to each other along the strap body;
the first strap end of the left abdominal strap being laterally fixed with the left trap band, offset from the at least one ring; and
the first strap end of the right abdominal strap being laterally fixed with the right trap band, offset from the at least one ring.

2. The adjustable posture resistance band as claimed in claim 1 comprising:
the left trap band and the right trap band each further comprising an inner band surface and an outer band surface;
the fastener mechanism comprising a lower fastener and an upper fastener;
the inner band surface being laterally positioned along the band body;
the outer band surface being laterally positioned along the band body, opposite to the inner band surface;
the lower fastener being fixed onto the inner band surface, adjacent to the first band end; and
the upper fastener being fixed onto the outer band surface, adjacent to the second band end.

3. The adjustable posture resistance band as claimed in claim 2 comprising:
the lower fastener comprising a lower interlocking tab member and a lower interlocking elongated member;
the lower interlocking tab member being fixed adjacent with the first band end;
the lower interlocking elongated member being positioned offset from the lower interlocking tab member;
the lower interlocking elongated member being fixed across the inner band surface;
the lower interlocking tab member of the left trap band being positioned through the at least one ring;
the lower interlocking tab member of the right trap band being positioned through the at least one ring; and
the lower interlocking tab member being detachably attached with the lower interlocking elongated member.

4. The adjustable posture resistance band as claimed in claim 3 comprising:
the lower fastener further comprising at least one tucking pocket;
the at least one tucking pocket comprising a first lateral edge, a second lateral edge, and a pocket panel;
the pocket panel being positioned adjacent with the inner band surface;
the pocket panel being positioned across the lower interlocking elongated member;
the first lateral edge and the second lateral edge being positioned opposite to each other across the pocket panel;
the first lateral edge being positioned adjacent and along the lower interlocking elongated member;
the second lateral edge being positioned adjacent and along the lower interlocking elongated member, opposite to the first lateral edge; and
the first lateral edge and the second lateral edge being fixed onto the inner band surface.

5. The adjustable posture resistance band as claimed in claim 3, wherein the lower interlocking tab member is a hooks portion of a hook-and-loop fastener, and wherein the lower interlocking elongated member is made of an elastic material.

6. The adjustable posture resistance band as claimed in claim 2 comprising:
the upper fastener comprising an upper interlocking tab member and an upper interlocking elongated member;
the upper interlocking tab member being fixed adjacent with the second band end;
the upper interlocking elongated member being positioned offset from the upper interlocking tab member;
the upper interlocking elongated member being fixed across the inner band surface;
the upper interlocking tab member of the left trap band being positioned through the at least one ring;
the upper interlocking tab member of the right trap band being positioned through the at least one ring; and
the upper interlocking tab member being detachably attached with the upper interlocking elongated member.

7. The adjustable posture resistance band as claimed in claim 6, wherein the upper interlocking tab member is a hooks portion of a hook-and-loop fastener, and wherein the upper interlocking elongated member is made of an elastic material.

8. The adjustable posture resistance band as claimed in claim 1 comprising:
the band body comprising a first linear portion, an ergonomic wider portion, and a second linear portion;
the ergonomic wider portion being fixed in between the first linear portion and the second linear portion;
the first band end being positioned coincident with the first linear portion, offset from the ergonomic wider portion; and
the second band end being positioned coincident with the second linear portion, offset from the ergonomic wider portion.

9. The adjustable posture resistance band as claimed in claim 8 comprising:
the first strap end of the left abdominal strap being fixed in between the first linear portion of the left trap band and the ergonomic wider portion of the left trap band.

10. The adjustable posture resistance band as claimed in claim 8 comprising:
the first strap end of the right abdominal strap being fixed in between the first linear portion of the right trap band and the ergonomic wider portion of the right trap band.

11. The adjustable posture resistance band as claimed in claim 1 comprising:
the left abdominal strap and the right abdominal strap each further comprising an inner strap surface, an outer strap surface, and a strap fastener;
the inner strap surface being laterally positioned along the strap body;
the outer strap surface being laterally positioned along the strap body, opposite to the inner strap surface;
the strap fastener of the left abdominal strap being fixed onto the inner strap surface of the left abdominal strap, adjacent to the second strap end;
the strap fastener of the right abdominal strap being fixed onto the outer strap surface of the right abdominal strap, adjacent to the second strap end;
the strap fastener of the left abdominal strap being detachably attached with the outer strap surface of the right abdominal strap; and
the strap fastener of the right abdominal strap being detachably attached with the outer strap surface of the left abdominal strap.

12. The adjustable posture resistance band as claimed in claim 1 comprising:
the left abdominal strap and the right abdominal strap each further comprising an inner strap surface, an outer strap surface, and a strap fastener;
the left trap band and the right trap band each further comprising an inner band surface and an outer band surface;
the inner strap surface being laterally positioned along the strap body;
the outer strap surface being laterally positioned along the strap body, opposite to the inner strap surface;
the inner band surface being laterally positioned along the band body;
the outer band surface being laterally positioned along the band body, opposite to the inner band surface;
the strap fastener of the left abdominal strap being fixed onto the inner strap surface of the left abdominal strap, adjacent to the second strap end;
the strap fastener of the right abdominal strap being fixed onto the inner strap surface of the right abdominal strap, adjacent to the second strap end;
the strap fastener of the left abdominal strap being detachably attached with the outer band surface of the right trap band; and
the strap fastener of the right abdominal strap being detachably attached with the outer band surface of the left trap band.

13. The adjustable posture resistance band as claimed in claim 1, wherein the at least one ring is a single ring.

14. The adjustable posture resistance band as claimed in claim 1 comprising:
a connecting strap;
the at least one ring comprising a left ring and a right ring;
the left ring being positioned adjacent with the connecting strap;
the right ring being positioned adjacent with the connecting strap, opposite the left ring;
the left ring and the right ring being tethered to each other by the connecting strap;
the first strap end of the left trap band and the second strap end of the left trap band being detachably attached to the left ring by the fastener mechanism of the left trap band; and
the first strap end of the right trap band and the second strap end of the right trap band being detachably attached to the right ring by the fastener mechanism of the right trap band.
